(19) United States
(12) Patent Application Publication
(10) Pub. No.: US 2002/0010197 A1
(43) Pub. Date: Jan. 24, 2002

(54) PHARMACEUTICAL COMPOSITIONS COMPRISING A COMPOUND HAVING DOPAMINE (D2) RECEPTOR AGONIST ACTIVITY AND A COMPOUND (B) HAVING BETA2-ADRENORECEPTOR AGONIST ACTIVITY

(76) Inventors: JOHN DIXON, MELTON MOWBRAY (GB); INCE FRANCIS, LOUGHBOROUGH (GB)

Correspondence Address: NIXON & VANDERHYE, 1100 NORTH GLEBE ROAD, 8TH FLOOR, ARLINGTON, VA, 222014714

(21) Appl. No.: 09/254,622

(22) PCT Filed: Dec. 22, 1998
(86) PCT No.: PCT/SE98/02427

(30) Foreign Application Priority Data
Jan. 13, 1998 (SE) 9800052-4
Feb. 5, 1998 (SE) 9800330-4

(51) Int. Cl. A61K 31/44; A01N 43/40

(52) U.S. Cl. 514/340

(57) ABSTRACT

The present invention provides novel pharmaceutical compositions and their use in the treatment of reversible obstructive airways diseases.

The present invention relates to pharmaceutical compositions and their use in the treatment of reversible obstructive airways diseases.

In accordance with the present invention, there is provided a pharmaceutical composition comprising a compound (A) having dopamine (D₂) receptor agonist activity and a compound (B) having β₂-adrenoreceptor agonist activity, wherein the compounds (A) and (B) are different.

In particular, the present invention provides a pharmaceutical composition comprising a compound (A) having dopamine (D₂) receptor agonist selected from the group consisting of:

Apomorphine ((R)-5,6,6a,7-tetrahydro-6-methyl-4H-dibenzo[de,g]quinoline-10,11-diol),

Bromocriptine ((5′α)-2-bromo-12′-hydroxy-2′-(1-methylethyl)-5′-(2-methylpropyl) ergotaman-3′,6′,18-trione),

Cabergoline ((8β)-N-[3-(dimethylamino)propyl]-N-[(ethylamino)carbonyl]-6-(2-propenyl)ergoline-8-carboxamide),

Lisuride (N′-[(8α)-9,10-didehydro-6-methylergolin-8-yl]-N,N-diethylurea),

Pergolide ((8β)-8-[(methylthio)methyl]-6-propylergoline),

Levodopa (3-hydroxy-L-tyrosine),

Pramipexole ((S)-4,5,6,7-tetrahydro-N⁶-propyl-2,6-benzothiazolediamine),

Quinpirole hydrochloride (trans-(−)-4aR-4,4a,5,6,7,8,8a,9-octahydro-5-propyl-1Hpyrazolo[3,4-g]quinoline hydrochloride),

Ropinirole (4-[2-(dipropylamino)ethyl]-1,3-dihydro-2H-indol-2-one) and

Talipexole (5,6,7,8-tetrahydro-6-(2-propenyl)-4H-thiazolo[4,5-d]azepin-2-amine) and

a compound (B) having β₂-adrenoreceptor agonist activity selected from the group consisting of:

Clenbuterol (4-amino-3,5-dichloro-α-[[(1,1-dimethylethyl)amino]methyl]benzenemethanol),

Fenoterol (5-[1-hydroxy-2-[[2-(4-hydroxyphenyl)-1-methylethyl]amino]ethyl]-1,3-benzenediol),

Formoterol ((±)-N-[2-hydroxy-5-[1-hydroxy-2-[[2-(4-methoxyphenyl)-1-methylethyl]amino]ethyl]phenylformamide),

[R,R]Formoterol,

Hexoprenaline (4,4′-[1,6-hexanediylbis[imino(1-hydroxy-2,1-ethanediyl)]]bis-1,2-benzenediol),

Isoetharine (4-[1-hydroxy-2-[(1-methylethyl)amino]butyl]-1,2-benzenediol),

Isoprenaline (4-[1-hydroxy-2-[(1-methylethyl)amino]ethyl]-1,2-benzenediol),

Metaproterenol (5-[1-hydroxy-2-[(1-methylethyl)amino]ethyl]-1,3-benzenediol),

Picumeterol (4-arnino-3,5-dichloro-α-[[[6-[2-(2-pyridinyl)ethoxy]hexyl]amino]methyl]benzenemethanol),

Pirbuterol (α⁶-[[(1,1-dimethylethyl)amino]methyl]-3-hydroxy-2,6-pyridinedimethanol),

Procaterol ((R*, S*)-(±)-8-hydroxy-5-[-hydroxy-2-[(1-methylethyl)amino]butyl]-2(1H)quinolinone),

Reproterol (7-[3-[[2-(3,5-dihydroxyphenyl)-2-hydroxyethyl]amino]propyl]-3,7-dihydro-1,3-dimethyl-1H-purine-2,6-dione),

Rimiterol (4-(hydroxy-2-piperidinylmethyl)-1,2-benzenediol),

Salbutamol ((±)-α¹-[[(1,1-dimethylethyl)amino]methyl]-4-hydroxy-1,3-benzenedimethanol),

[R]-Salbutamol,

Salmeterol ((±)-4-hydroxy-α¹-[[[6-(4-phenylbutoxy)hexyl]amino]methyl]-1,3-benzenedimethanol),

[R]-Salmeterol,

Terbutaline (5-[2-[(1,1-dimethylethyl)amino]-1-hydroxyethyl]-1,3-benzenediol),

Tulobuterol (2-chloro-α-[[(1,1-dimethylethyl)-amino]methyl]benzenemethanol) and

TA-2005 (8-hydroxy-5-[(1R)-1-hydroxy-2-[N-[(1R)-2-(4-methoxyphenyl)-1-methylethyl]amino]ethyl]carbostyril hydrochloride).

The compounds (A) and (B) above are known to be used separately as pharmaceuticals but the use of a compound (A) in combination with a compound (B) in a pharmaceutical composition is not known.

Certain compounds (A) and (B) are capable of existing in stereoisomeric forms. Unless otherwise indicated, it should be understood that the invention encompasses the use of all geometric and optical isomers of compounds (A) or of compounds (B), and mixtures thereof including racemates. The use of tautomers and mixtures thereof also form an aspect of the present invention.

Preferably the composition comprises, as compound (A), cabergoline or ropinirole.

The composition preferably comprises, as compound (B), formoterol, [R,R]-formoterol, salmeterol, [R-]-salmeterol, [R]-salbutamol or terbutaline.

The pharmaceutical composition of the invention may be prepared by mixing a compound (A) with a compound (B). Therefore, in another aspect of the present invention, there is provided a process for the preparation of a pharmaceutical composition which comprises mixing a compound (A) with a compound (B) as hereinbefore defined. The pharmaceutical composition of the invention may, and indeed will usually, contain various other ingredients known in the art, for example, a carrier, binder, lubricant, diluent, stabilising agent, buffering agent, emulsifying agent, viscosity-regulating agent, surfactant, preservative, flavouring or colorant. Thus the pharmaceutical composition of the invention will typically comprise a total amount of compound (A) and compound (B) (the active ingredients) in the range from 0.05 to 99%w (per cent by weight), more preferably in the range from 0.10 to 70%w, all percentages by weight being based on total composition.

The pharmaceutical compositions of the present invention have both β₂-adrenoreceptor agonist activity and dopamine (D₂) receptor agonist activity. β₂-Adrenoreceptor agonist activity may be determined in a test carried out on the isolated trachea of the guinea pig according to the method of I. G. Dougall et al., Br. J. Pharmacol., 1991, 104, 1057. Dopamine (D₂) receptor agonist activity may be assessed by the binding affinities of compounds for the dopamine receptor binding sites in bovine pituitary membranes according to the method of D. R. Sibley et al., J. Biol. Chem., 1982, 257(11), 6351-6361, or, in the functional rabbit isolated ear artery screen described by R. Brown et al., Br. J. Pharmacol., 1981, 73, 189P.

The present pharmaceutical compositions are particularly suitable for use in the treatment of reversible obstructive airways diseases such as asthma (including bronchial asthma, allergic asthma and intrinsic asthma, e.g. late asthma and airway hyper-responsiveness), chronic bronchitis and other chronic obstructive pulmonary diseases.

Thus, the present invention further provides a pharmaceutical composition as hereinbefore defined for use in therapy.

In a further aspect, there is provided the use of a pharmaceutical composition as hereinbefore defined in the manufacture of a medicament for the treatment of reversible obstructive airways disease, in particular for the treatment of asthma or chronic bronchitis.

The present invention still further provides a method of treating, or reducing the risk of, a reversible obstructive airways disease in a patient suffering from, or at risk of, said disease, which comprises administering to the patient a therapeutically effective amount of a pharmaceutical composition as hereinbefore defined.

For the above-mentioned therapeutic uses the dosage administered will, of course, vary with the compounds (A) and (B) employed, the mode of administration, the treatment desired and the disorder indicated. However, in general, satisfactory results will be obtained when the pharmaceutical composition is administered such that the total daily dosage of compound (A) and compound (B) together is in the range from 5 to 1500 μg, e.g. from 10 to 1450 μg or from 20 to 1400 μg.

The pharmaceutical composition of the invention may be administered topically (to the lung and/or airways) in the form of solutions, suspensions, aerosols and dry powder formulations; or systemically, e.g. by oral administration in the form of tablets, capsules, syrups, powders or granules, or by parenteral administration in the form of solutions or suspensions.

For example metered dose inhaler devices may be used to administer the active ingredients, dispersed in a suitable propellant and with or without additional excipients such as ethanol, surfactants, lubricants or stabilising agents.

Suitable propellants include hydrocarbon, chlorofluorocarbon and hydrofluoroalkane (e.g. heptafluoroalkane) propellants, or mixtures of any such propellants. Especially preferred propellants are P134a and P227, each of which may be used alone or in combination with other propellants and/or surfactants and/or other excipients.

Nebulised aqueous suspensions or, preferably, solutions may also be employed, with or without a suitable pH and/or tonicity adjustment, either as a unit-dose or multi-dose formulations.

Dry powder inhalers may be used to administer the active ingredients, alone or in combination with a pharmaceutically-acceptable carrier, in the latter case either as a finely divided powder or as an ordered mixture. The dry powder inhaler may be single dose or multi-dose and may utilise a dry powder or a powder-containing capsule.

Metered dose inhaler, nebuliser and dry powder inhaler devices are well known and a variety of such devices are available.

Tablets and gelatin capsules, which may be coated if desired, containing the active ingredients may, for example, also include one or more diluents, carriers, binders, lubricants or stabilising agents.

Injectable solutions of the active ingredients may also contain, for example, one or more preservatives, stabilising agents, viscosity-regulating agents, emulsifying agents or buffering agents.

1. A pharmaceutical composition comprising a compound (A) having dopamine (D₂) receptor agonist activity and a compound (B) having β₂-adrenoreceptor agonist activity, wherein the compounds (A) and (B) are different.

2. A composition according to claim 1 comprising a compound (A) having dopamine (D₂) receptor agonist activity selected from the group consisting of apomorphine, bromocriptine, cabergoline, lisuride, pergolide, levodopa, pramipexole, quinpirole hydrochloride, ropinirole and talipexole, and a compound (B) having β₂-adrenoreceptor agonist activity selected from the group consisting of clenbuterol, fenoterol, formoterol, [R,R]-formoterol, hexoprenaline, isoetharine, isoprenaline, metaproterenol, picumeterol, pirbuterol, procaterol, reproterol, rimiterol, salbutamol, [R]-salbutamol, salmeterol, [R]salmeterol, terbutaline, tulobuterol and TA-2005.

3. A composition according to claim 2, wherein, as compound (A), cabergoline or ropinirole is used.

4. A composition according to claim 2, wherein, as compound (B), formoterol, [R,R]-formoterol, salmeterol, [R-]-salmeterol, [R]-salbutamol or terbutaline is used.

5. A pharmaceutical composition as claimed in any one of claims 1 to 4 for use in therapy.

6. Use of a pharmaceutical composition as claimed in any one of claims 1 to 4 in the manufacture of a medicament for the treatment of reversible obstructive airways disease.

7. A method of treating, or reducing the risk of, a reversible obstructive airways disease in a patient suffering from, or at risk of, said disease, which comprises administering to the patient a therapeutically effective amount of a pharmaceutical composition as defined in any one of claims 1 to 4.
